(19) United States
(12) Patent Application Publication
(10) Pub. No.: US 2006/0253197 A1
(43) Pub. Date: Nov. 9, 2006

(54) Shape-memory port-access tube

(76) Inventor: Bradford NaPier, Honolulu, HI (US)

Correspondence Address: Paradise Patent Services, Inc.;Attn: George Darby, P.O. Box 893010, Mililani, HI, 96789-3010, US

(21) Appl. No.: 11/430,387

(22) Filed: May 8, 2006

Related U.S. Application Data
(60) Provisional application No. 60/679,582, filed on May 9, 2005.

(51) Int. Cl. A61F 2/20; A61F 2/04

(52) U.S. Cl. 623/9; 623/23.7

(57) ABSTRACT

The NaPier tube is a self-expanding port-access tube for insertion into body passages. A NaPier tube is typically used with an endoscope disposed within the lumen of an unactivated tube to enable visual navigation to place the tube in a passage. A NaPier tube comprises a shape-memory element, wall material, and a removable sheath. A tear-away, removable sheath maintains the shape-memory element in a compressed state. Upon placement of the distal end of a NaPier tube in the target location in a passage, the sheath is ruptured and removed through the port and the shape-memory element expands to its memorized geometries. Embodiments of the NaPier tube are adapted by length and memorized dimensions for endotracheal intubation and for port-access procedures, such as endoscopic surgery.

### BACKGROUND OF THE INVENTION

1. Technical Field

This invention relates to shape-memory tubes that are inserted, while compressed and sheathed, through a natural or surgical port into a natural or surgical passage in humans and animals; after placement in a passage, the compressing sheath is removed and the shape-memory tube of the invention expands to its memorized diameter. In an endotracheal embodiment of the invention, after placement of a shape-memory tube from the mouth or nose of a patient to the patient's trachea, the tube is expanded to its memorized diameter and the lumen of the expanded tube is used to facilitate respiration, observation, medication (including anesthesia), surgery, and/or therapy. Navigation of a shape-memory tube during insertion and placement is typically guided by an endoscope disposed in the lumen of the tube. The tube is easily removed after use.

2. Description of the Related Art

A primary object (i.e., technical problem to be solved) of the invention is an improved apparatus and method of endotracheal intubation. Endotracheal intubation is a common medical procedure used for examination, diagnosis, anesthesia, and surgery. Endotracheal intubation can be problematic when a patient has a limited airway secondary to congenital problems, anatomic blockage (e.g., large tonsils; redundant pharyngeal mucosa; prolapse of the tongue base; tumors of larynx, pharynx or hypopharynx; post radiation edema of the pharynx), infective processes (e.g., epiglottitis, retropharyngeal abscess, or prevertebral abscess), medical conditions (e.g., obesity, general anesthesia by IV), or existing medical devices (e.g., neurosurgical stability devices, cervical collars, and halo devices).

In preparation for a major surgical procedure, patients are typically administered 100% oxygen for a few minutes using a face mask, then one or more anesthetics are administered intravenously. As soon as the anesthetics take effect, an endotracheal tube is put in place from the patient's mouth (proximal end) to the trachea (distal end). As a practical matter, the anesthesiologist has a goal of taking two minutes or less to insert an endotracheal tube into the trachea of an anesthetized patient, inflating the expandable tracheal cuff on the tube, hooking up the fitting at the proximal end of the tube to a mechanical respirator, and commencing positive pressure breathing (mechanical ventilation) of the patient. After about two minutes from the time of administration of anesthesia, if mechanical ventilation has not begun, asphyxia begins. If positive pressure respiration (mechanical ventilation) of the patient is not achieved within the initial two minutes, an antidote to the anesthetic is intravenously administered to the patient so that normal breathing will commence before damage from asphyxia (e.g., brain damage) can occur. After a failed intubation and a revival by antidote, surgery must be postponed, usually for twenty-four hours or more. Rescheduled surgeries incur a large administrative, professional, and emotional cost. Moreover, cases of asphyxia after a failed intubation still occur.

The term “body passage” or “passage” means herein an internal body lumen that communicates directly or indirectly with a proximal opening to the external environment, e.g., the respiratory tract (broadly, oral and nasal cavities, pharynx, larynx, trachea, bronchia), the alimentary canal, a ureter, urethra, prostate, colon, uterus, vagina, ear canal, Eustachian tube, ducts, etc. The term “body passage” or “passage” also includes surgically created lumens communicating with a proximal opening to the external environment, e.g., passages created in laparoscopic surgery such as closed-chest (port-access) thoracoscopic bypass surgery and cholecystectomy. The term “port” means the opening of a body passage to the external environment. The term “port-access” means the use of a tube inserted through a port to access a passage in communication with the port. “Axis” means the longitudinal axis of the port-access tube of the invention. “Operator” means a healthcare professional or other person performing an intubation or other port-access procedure using an embodiment of the invention. The term “memorized diameter” means the austenitic diameter of a round tube made with a shape-memory alloy. The term “memorized geometry” means the austenitic dimensions of a tube made with a shape-memory alloy, which geometry may include angles. When compressed while at a temperature above the austenite finish temperature, a tube made of shape-memory alloy transitions to a martensite phase, and when the compressive force is removed, e.g., by removing a sheath restraining a compressed tube, the tube of shape-memory alloy expands to its memorized, or austenitic, diameter or geometry. “Shape-memory”, as an adjective, means made of, or related to, a shape-memory material, typically a metal alloy such as nickel/titanium, but also including shape-memory polymers and stainless steel (e.g., wire, braid, lattice-cut). Although Cu/Zn/Al and Cu/Al/Ni shape-memory alloys can be used in the invention, the properties of Ni/Ti alloys are superior. Embodiments using stainless steel wire are less expensive than embodiments using nitinol. Nitinol alloys, properly processed, can exhibit their optimum shape-memory behavior at human body temperatures. “Shape-memory element” means a tube made of a shape-memory material.

Even in non-obese patients, general anesthesia induced by intravenous injection can cause the pharyngeal, hypopharyngeal, and laryngeal regions to become flaccid, which makes intubation (for ventilation and for delivery of nebulized anesthetic) problematic. In obese patients, the problem of flaccid airways secondary to general anesthesia by IV is particularly acute. Many patients, typically those who are overweight or have short necks, cannot move their head so that the operator can see a patient's vocal cords when the operator looks into a patient's mouth. The passage from the nose or mouth to the trachea may be small, limited, or tortuous. Placement of a traditional, rigid, oral endotracheal tube, such as commonly available endotracheal tubes made by MALLINCKRODT®, SHERIDAN®, RUSCH®, or PORTEX® (collectively called, “traditional endotracheal tubes”), in small, limited, or tortuous passages may be difficult or dangerous; the heightened risk in some cases leaves no alternative but a tracheostomy. Endotracheal tube length and diameter are selected based on the patient to be intubated. A large adult male may require a 30 cm length and 8 mm inside diameter endotracheal tube. Shorter, smaller diameter tubes are used for juveniles and infants.

Most endotracheal tubes have an expandable cuff near the distal end of the tube. The cuff at the distal end is in gaseous or fluid communication with a connector at the proximal end of the tube. Upon placement in the trachea, the cuff is inflated and presses against the tracheal lining to prevent bypass of the tube, i.e., inhalation and exhalation is solely through the lumen of the tube after inflation of the cuff.

Traditional endotracheal tubes reflect a compromise in the selection of the diameter of the tube: smaller diameter tubes are easier to insert and inflict less damage to the tissue lining of the passage, but the smaller lumen diameters of such tubes and have greater airway resistance and therefore inhibit air movement into and out of the lungs; larger diameter tubes are harder to insert (and may be blocked in limited airways), are more likely to damage the tissue lining of the passage during placement, but significantly improve air movement into and out of the lungs. Traditional endotracheal tubes have an average wall thickness of 1 to 2 mm, which means that about 2 to 4 mm of the overall diameter of a tube is consumed by the structure and is unavailable for ventilation.

Natural passages other than the oratracheal or nasotracheal passages are involved in medical examination, diagnosis, anesthesia, surgery, and therapy, and also can be small, limited, or tortuous. Other objects of the invention are improved intubation apparatus and methods for natural passages other than oratracheal or nasotracheal, e.g., alimentary canal (accessed from the mouth or anus), ureter, urethra, prostate, uterus, ear canal, Eustachian tube, etc. For example, embodiments of the invention can be used where anatomic, infectious, or other problems make traditional drainage catheters, such as a urethral catheter, too risky. Another object of the invention is for use in port-access surgery, e.g., closed-chest coronary bypass surgery and cholecystectomy.

Although the related art contains expandable endotracheal tubes, such as those described in U.S. Pat. No. 4,141,364 to Schultze, previous expandable endotracheal tubes and cannulas have required a ram, rod, balloon, pressurized fluid, pressurized gas, or other means to expand the tube. The inflation of the balloon portion of a dilation catheter positioned in the lumen of the unexpanded tube, or the insertion from proximal to distal end of a larger diameter conical rod into the lumen of the unexpanded tube, or the introduction of pressurized gas or fluid into channels embedded in wall of the unexpanded tube, expands compressed endotracheal tubes of the existing art to larger diameter tubes. Such additional apparatus or means adds complexity and potential points of failure to intubation, and does not solve the problem of guidance of a port-access tube through limited passages. The prior art lacks any enabling disclosure of a radially self-expanding endotracheal tube or port-access tube.

There is voluminous art related to endovascular stents, including polymer-coated endovascular stents using a nitinol frame, such as disclosed in US Patent Application 20040116946 (Goldsteen). Some endovascular stents are self-expanding, such as those described in U.S. Pat. No. 6,860,898 (Stack). Details of prior art expandable stents and stent-grafts can be found in Stack, and in U.S. Pat. No. 3,868,956 (Alfidi); U.S. Pat. No. 4,512,338 (Balko); U.S. Pat. No. 4,553,545 (Maass); U.S. Pat. No. 4,733,665 (Palmaz); U.S. Pat. No. 4,762,128 (Rosenbluth); U.S. Pat. No. 4,800,882 (Gianturco); U.S. Pat. No. 5,514,154 (Lau, et al.); U.S. Pat. No. 5,421,955 (Lau); U.S. Pat. No. 5,603,721 (Lau); U.S. Pat. No. 4,655,772 (Wallsten); U.S. Pat. No. 4,739,762 (Palmaz); U.S. Pat. No. 4,580,568 (Gianturco); U.S. Pat. No. 4,830,003 (Wolff); U.S. Pat. No. 5,569,295 (Lam), U.S. Pat. No. 5,628,788 (Pinchuk), and U.S. Pat. No. 6,569,191 (Hogan), which are hereby incorporated by reference. As explained below, there are substantive structural, functional, and design differences between stents and stent-grafts (collectively, “stents”) on the one hand, and port-access tubes on the other hand.

All embodiments of the tube of the present invention are self-expanding, and therefore share one, and only one, attribute, self-expansion, with self-expanding stents. Otherwise, the art of the present invention differs substantially from the art of self-expanding stents in the areas of wall construction, external interfaces, delivery system, activation system, degree of expansion, strength, dimensions, the inclusion of ducts (e.g., inflation duct, dispensing duct, suction duct) and additional lumens (e.g., for lighting systems, surgical instruments), and removability. All stents enlarge the diameter of stenotic vessels and typically have a porous annular wall to permit infiltration by endothelial cells; infiltration of endothelial cells anchors the stent permanently in place. Port-access tubes, such as endotracheal tubes, have a non-porous, airtight, waterproof annular wall, and are always removed after use. Some stents are simple spirals or braids of nitinol with no wall. If stents are coated with an elastomer, the elastomer is very porous and is typically used to elute drugs loaded in coating porosities to the tissue surrounding the activated stent. In contrast, the elastomeric coating or annular fabric of the present invention has minimal porosity and essentially no permeability to prevent infiltration by endothelial cells and leakage of gas or fluid.

Stents do not interface with the external environment; after deployment, they remain inside a patient's body. Port-access tubes always communicate with the external environment, and typically have fittings on the proximal end for connection to pumps, dispensers, endoscopes, sensors, etc.

All stents require a separate and detachable delivery system, e.g., an endovascular catheter system and an external radiographic monitoring system. Port-access tubes have an integral delivery system, i.e., the distal portion and proximal portion of a port-access tube cannot be separated or detached; this also facilitates easy removal of a tube. For activation, many stents require inflation of a catheter-born balloon or other source of radial force to expand (activate) the stent from compressed or “placement” diameter to expanded or “activated” diameter. Self-expanding stents require very complicated, multi-element, catheter-based activation systems designed to inhibit forward movement of the catheter tip and to inhibit “jumping” of the stent while a proximal lever or grip is squeezed to retract the jacket surrounding the stent. In contrast, the tube of the present invention has a simple, tear-away sheath activation system with far fewer activation elements and no separate delivery elements.

Although self-expanding stents and the shape-memory port-access tube of the present invention both use shape-memory metals, such as nitinol, to provide a radial force to expand the annular wall after placement, port-access tube alloy composition and degree of expansion of the annular wall from placement diameter to activated diameter are selected to provide a much less compressible tube wall after activation compared with the walls of stents. An endotracheal tube, for instance, must withstand the compressive force of an inflatable cuff on the annular wall, and inadvertent compressive forces on the airway in the throat region, e.g., impact to the throat during movement of an intubated accident or combat casualty. Stents, in contrast, are designed to be flexible during placement to permit navigation of small blood vessels, and very flexible after activation to avoid rupture of the vessel lining.

Finally, since all port-access tubes are removed after use, tubes can contain special features to facilitate removal, which features are totally absent in stents. In summary, self-expansion is only one of a constellation of considerations in the design and enablement of a self-expanding endotracheal tube or other port-access tube, just as expansion is only one of a constellation of considerations in the design and enablement of dilation catheters.

There is demand for a lower risk apparatus and method of endotracheal intubation, especially as an alternative to tracheostomy and to avoid the cost of failed intubations. There is also an unmet demand for a lower risk apparatus and method of intubation for use in port-access surgery (e.g., as a cannula after opening a passage with a trocar or scalpel) and for use in natural passages (e.g., as a catheter or cannula) in addition to the endotracheal passage. This unmet need is particularly critical if endotracheal intubation must be performed outside an operating room or by persons, such as emergency medical technicians, paramedics, military medics, and physicians who do not routinely intubate patients; the demand for an improved device is critical if the patient has a limited airway. The existing art of expanding endotracheal tubes has not solved this problem. There is also demand for an apparatus for intubation and port-access procedures that better accommodates intra-lumen, endoscopic navigation.

### SUMMARY OF THE INVENTION

The apparatus of the invention, called herein the “NaPier tube”, in a round lumen embodiment, is a circumferentially (i.e., radially about the longitudinal axis) self-expanding port-access tube for insertion into body passages. A NaPier tube is typically used with an endoscope disposed within the lumen of an unactivated tube to enable visual navigation of a passage. An embodiment of the NaPier tube for endotracheal intubation is called a “NaPier ET tube.” The length and memorized diameter of a NaPier ET tube depends upon the length and width of the respiratory tract to the intubated; such dimensions are well known in the art. An embodiment of the NaPier tube for port-access procedures is called a “NaPier port-access tube”. The term “NaPier tube” includes both NaPier ET tubes and NaPier port-access tubes. One embodiment of a NaPier tube is open at both ends and has an unobstructed lumen; the annular edge of the distal end (the end placed deepest in a passage) is smooth to facilitate insertion. The proximal end of a NaPier tube may be plain or may have a hub with specialized fittings (a “hubbed tube”), such as a connector for a mechanical ventilator in the case of an endotracheal tube for use in an operating room.

The structure of a preferred embodiment of an unactivated NaPier tube comprises a compressed, round, tubular, shape-memory element in circumferential association with a thin, expandable, elastomeric or fabric element. An unactivated NaPier tube has a restraining means, typically a removable, tear-away sheath, to maintain the shape-memory element in a compressed state; the restraining means extends the full-length of a plain tube, or from the distal end to the hub of a hubbed tube to the distal end of the tube. Upon placement of the distal end of a NaPier tube in the target location, the tear-away sheath is removed, which permits the shape-memory element to expand to its memorized diameter or until resistance from contact with the passage lining restrains further expansion of the activated shape-memory element. The shape-memory element used in a NaPier tube is typically nitinol, a shape-memory alloy of nickel and titanium that can be manufactured as a braid, wire, ribbon, or laser-cut tube, imparted with a memorized diameter, coated with wall material, compressed to the unactivated diameter, and placed within a tear-away sheath.

To use the invention, an unactivated NaPier tube is inserted by an operator into the proximal opening (“port”) of a passage (e.g., anterior nares, in the case of a NaPier endotracheal tube being placed for nasotracheal intubation), and progressively introduced into the passage until the distal end of the NaPier tube reaches the desired location in the passage (e.g., in the trachea for endotracheal intubation). The tear-away sheath is then removed, in a preferred embodiment by using two levers integral with the proximal end of the tear-away sheath that, when operated, rupture a seam between halves of the tear-away sheath. The ruptured halves of the tear-away sheath are withdrawn from the passage by the operator's withdrawing each longitudinal half-sheath through the port while the hub (or proximal end of the tube) is stationary. The removal of the tear-away sheath activates the tube (permits the shape-memory element to expand). The activation (expansion) of the shape-memory element forces the expandable elastomer or fabric element circumferentially associated with the shape-memory element to stretch to the memorized diameter without openings in the wall material of an activated (expanded) tube.

“Wall material” means the expandable elastomer or fabric element on the exterior, the interior, or the interior and exterior sides of a shape-memory element. Shape-memory port-access tubes are typically round, but the memorized geometry of the shape-memory material can be any tubular shape, e.g., square, rectangular, triangular, etc., and a single shape-memory port-access tube can have one or more memorized geometries along its length. “Removable sheath” and “sheath” (other than a “removal sheath”, as defined below) mean a tear-away sheath or a pull-string sheath, as defined below, or other sheath known in the art that can be removed after final placement of the sheathed tube; the removal of a removable sheath activates the shape-memory element. Only one removable sheath is needed on a given NaPier tube.

A NaPier tube is typically used in conjunction with an endoscope with an outside diameter small enough to be inserted in the axial lumen of an unactivated NaPier tube. For instance, a 1.8 mm (e.g., for pediatric endoscopy) to 3.8 mm (e.g., for adult endoscopy) outside diameter bronchoscope is used with NaPier endotracheal tubes having a slightly larger axial lumen diameter than the bronchoscope's outside diameter. Before insertion of the NaPier tube into a passage, the operator inserts the endoscope into the axial lumen of the NaPier tube until the distal end of the endoscope is aligned with the distal end of the NaPier tube. The operator inserts the NaPier tube into the proximal opening of the passage, manipulates the navigational controls on the proximal end of the endoscope based on the images of the passage provided by the endoscope, and progressively feeds the tube into the passage until the distal ends of the endoscope and NaPier tube, still aligned, reach the desired location (“target”) in the passage. The NaPier tube is activated and the endoscope is then typically withdrawn. The activated NaPier tube is used for examination, diagnosis, anesthesia, surgery, therapy, or other procedure, and after completion of the procedure(s), the operator withdraws the NaPier tube from the passage by pulling on the proximal end. Pulling on the proximal end of the tube generates an elongating tensile force that causes the shape-memory element to reduce its diameter, which facilitates withdrawal of the tube.

Various embodiments of the NaPier tube are adapted for use in specific passages. For instance, a NaPier ET tube can be made with an inflatable cuff near the distal end; when inflated by a pressurized gas or fluid introduced into a longitudinal “inflation duct” between the proximal end and the cuff, the cuff expands to provide an airtight, tracheal seal around the activated tube. A NaPier ET tube with an inflatable cuff in the distal portion of the tube is called an “inflatable cuff” NaPier ET tube. Alternatively, rather than using inflation to activate a cuff, the cuff can be formed by imparting a larger memorized diameter to a distal portion of a round shape-memory element; when the tube is activated, such distal portion expands until the inner wall of the trachea prevents further expansion, thereby providing a substantially airtight seal. A NaPier ET tube with such a larger memorized diameter in the distal portion of the tube is called an “integral cuff” NaPier ET tube. A NaPier ET tube without a cuff is called a “cuffless” NaPier ET tube. A NaPier ET tube can also be made with a longitudinal duct (“dispensing duct”) between a hub at the proximal end and a point near the distal end for administration of nebulized anesthetic and/or medicament. A NaPier ET tube typically has an unactivated axial lumen diameter of less than 4 mm, is used with a bronchoscope with outside diameter slightly smaller than the axial lumen diameter of the unactivated NaPier ET tube, and upon activation the tube (in an embodiment for use in adults) expands to an axial lumen diameter of about 7 mm and outside diameter of about 8 mm. After activation of a NaPier ET tube, the lumen of the NaPier ET tube (i.e., the airway) does not collapse when a ventilator is attached to the proximal end of the NaPier ET tube.

The NaPier ET tube enables much less traumatic placement of an endotracheal tube as a result of using a smaller diameter, unactivated tube, and especially when placed using direct (endoscopic) vision. The NaPier ET tube provides improved ability to traverse limited airways in young children and infants, or secondary to tumors, infection, or abnormal anatomy. For nasotracheal intubation, if a NaPier ET tube is used, there is no advancement of a large endotracheal tube through the nose before or after placement of the bronchoscope in the trachea. The NaPier ET tube is less traumatic to the nasal passage and provides a safer deployment; there is greatly reduced risk of bleeding and of blood obstructing the airway during placement. An unactivated NaPier ET tube is much easier to place than a standard endotracheal tube during oratracheal or, especially, during nasotracheal intubation. This means that placement in a limited passage can be done by an operator with less training than an otolaryngologist, anesthetist, pulmonologist, or other specialist. For instance, an emergency medical technician or combat medic could use a NaPier ET tube rather than perform a tracheostomy, which permanently scars the patient's throat. The NaPier ET tube satisfies the demand for a lower risk apparatus and method of endotracheal intubation, especially as an alternative to tracheostomy and to avoid the cost of failed intubations.

The NaPier tube can be selected to expand to the exact outer diameter of a standard endotracheal tube, but the thinner walls of the expandable endotracheal tube provide a larger lumen (the wall of the NaPier ET tube is typically less than half the thickness, but with no compromise in strength, compared with a standard endotracheal tube); this represents a significant advance in the art of endotracheal tubes. The larger tube lumen provided by the NaPier ET tube has reduced resistance to respiratory airflow, and the increased lumen area provides more space for insertion of instruments through the tube after the tube is activated, compared with standard endotracheal tubes.

### BRIEF DESCRIPTION OF THE DRAWINGS

FIG. 1 is a three-quarter perspective view of a hubbed, unactivated NaPier tube.

FIG. 2 is a front view of the hub of a hubbed, tear-away sheath, unactivated NaPier tube, showing scoring on one side of the sheath.

FIG. 3 is a longitudinal cross-section view of the proximal portion of a hubbed, unactivated NaPier tube.

FIG. 4 is a diagram of an unactivated, hubbed, tear-away sheath, NaPier ET tube inserted in the trachea.

FIG. 5 is a diagram of a hubbed, tear-away sheath, NaPier ET tube inserted in the trachea, after twisting of the sheath flanges to rupture the sheath and begin activation.

FIG. 6 is a diagram of a hubbed, tear-away sheath, cuffless, NaPier ET tube inserted in the trachea, after about half of the sheath has been ruptured and withdrawn through the port, with activation of a corresponding length of the shape-memory element.

FIG. 7 is a longitudinal cross-section view of the proximal portion of a hubbed, activated NaPier tube.

FIG. 8 is a diagram of a hubbed, cuffless, NaPier ET tube inserted in the trachea, after activation of the entire length of the shape-memory element.

FIG. 9 is a diagram of a hubbed, integral cuff, NaPier ET tube inserted in the trachea, after activation of the entire length of the shape-memory element.

FIG. 10 is a diagram of a hubbed, inflatable cuff, NaPier ET tube inserted in the trachea, after activation of the entire length of the shape-memory element and inflation of the inflatable cuff.

FIG. 11 is a diagram of a hubbed, inflatable cuffs, double-lumen, NaPier ET tube inserted in the trachea and one bronchus, after activation of the entire length of the shape-memory element, and inflation of the tracheal inflatable cuff and of a bronchial inflatable cuff.

### DETAILED DESCRIPTION OF THE PREFERRED EMBODIMENTS

An embodiment of the NaPier tube with a round lumen after activation (“round-lumen embodiment’) is a circumferentially (i.e., radially about the longitudinal axis) self-expanding port-access tube for insertion through a port into a body passage. A basic, unactivated embodiment of a NaPier tube comprises a compressed, round, tubular, shape-memory element (“frame”) in circumferential association with a thin, expandable, elastomeric or fabric wall material and contained in a removable sheath. The removable sheath maintains the shape-memory element in a compressed state; the sheath extends around the exterior surface of the full-length of a plain tube or from the distal end to the hub, defined below, of a hubbed tube.

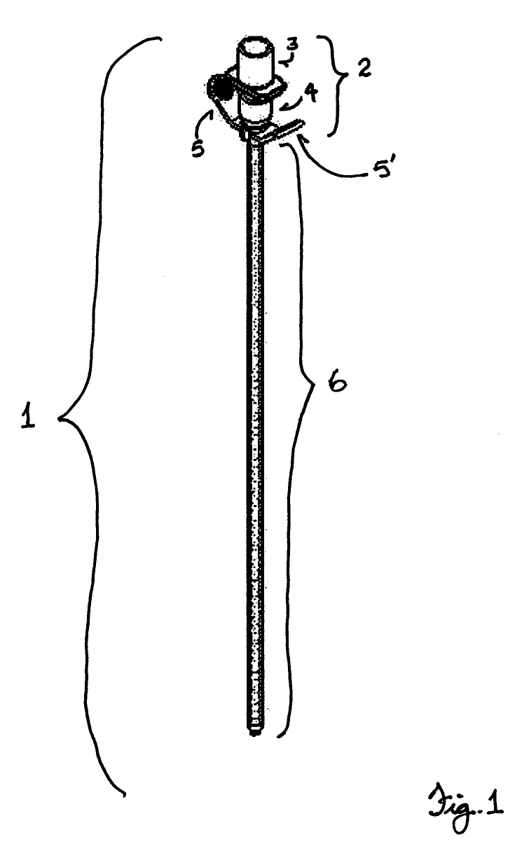

FIG. 1 shows an unactivated, hubbed NaPier tube (1) with a preferred embodiment of a hub (2) and a tear-away sheath. A fitting (3) is solidly affixed to a sleeve (4) that has external threads on its distal portion. The fitting (3) and sleeve (4) form the hub (2). In a NaPier ET tube, the fitting (3) is a hose fitting for connection to a mechanical ventilator. The hub of a hubbed NaPier tube is typically made of medical-quality plastic and equipped with fittings and other optional features described below (collectively, “fittings”). The hub is joined to the proximal end of the frame and has an airtight seal with the wall material. Threads on the internal, proximal surface of a pair of sheath flanges (5, 5′) engage the threads on the sleeve (4). Each sheath flange is affixed to the proximal portion of a half-circumference of the tear-away sheath (6). The sheath has full-length, longitudinal scoring at the two points where the two flanges abut each other. The sleeve can be formed with a knurled surface (not shown) to provide a better grip. The tear-away sheath can optionally be made with more than two sheath flanges and corresponding longitudinal scorings.

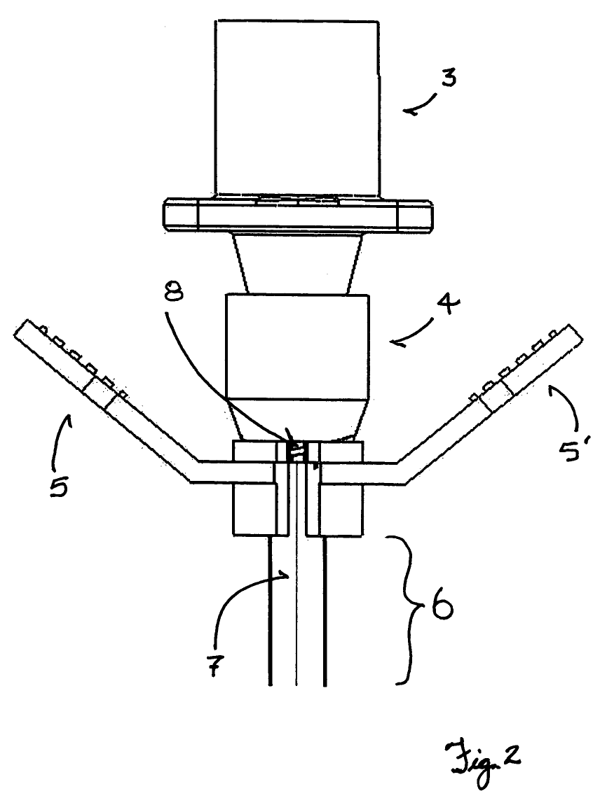

FIG. 2 shows a front view of the hub of a hubbed, unactivated, tear-away sheath, NaPier tube, with scoring (7) on the visible side of the sheath. The opposite side of the sheath (6) is similarly scored. Threads (8) are on the external, distal portion of the sleeve (4). When an operator rotates the sheath flanges (5, 5′) against a stationary hub (3 and 4 combined), or alternatively, rotates the hub against stationary sheath flanges, for approximately 180 degrees, the threads (8) on the distal portion of the sleeve (4) advance into to the sheath flange threads, and the resultant radial motion of the flanges being displaced by the distal portion of the sleeve ruptures the sheath along the scorings on the sheath.

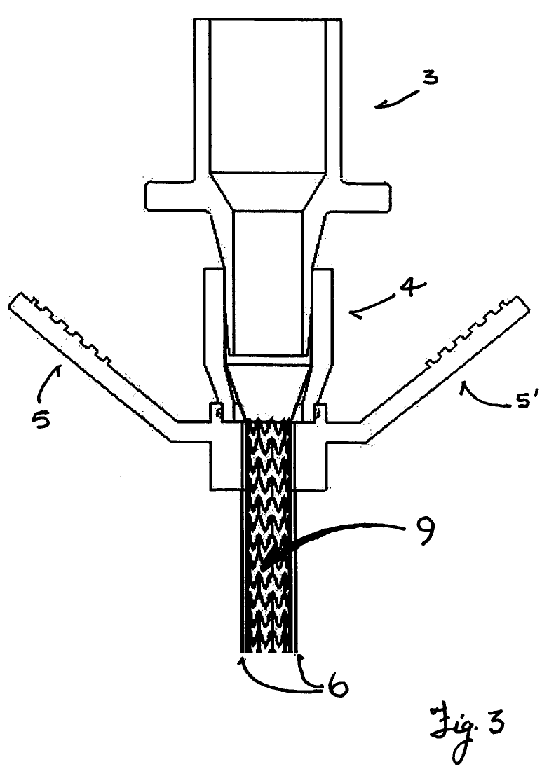

FIG. 3 shows a longitudinal cross-section view of the proximal portion of a hubbed, unactivated NaPier tube. Laser-cut shape shape-memory material (9) is disposed in a compressed, martensitic state inside the sheath (6).

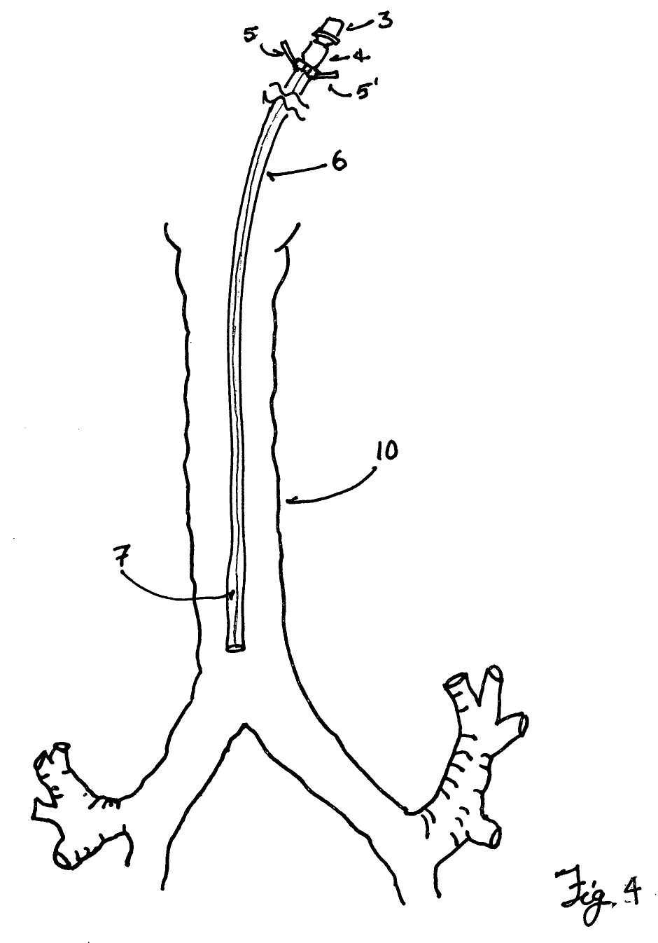

FIG. 4 shows a hubbed, tear-away sheath, NaPier ET tube inserted in the trachea (10) (insertion could have been done without the use of an endoscope, or with endoscope navigation). The sheath flanges (5,5′) have not been rotated against the hub (3 and 4), therefore the scoring (7) is not yet ruptured and the sleeve (6) is intact.

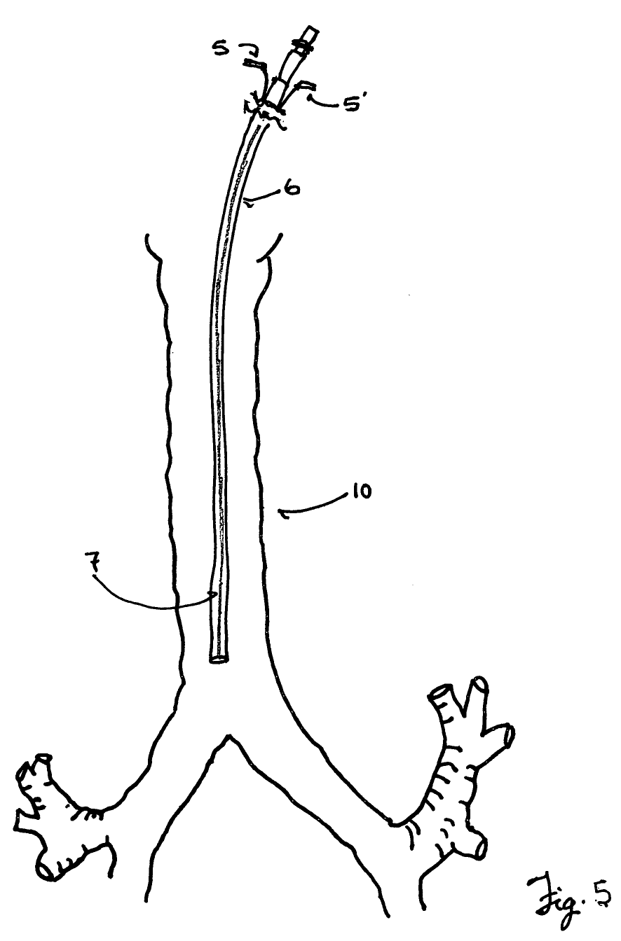

FIG. 5 shows a hubbed, tear-away sheath, NaPier ET tube inserted in the trachea (10) after the sheath flanges (5,5′) have been rotated against the hub (3 and 4), thereby rupturing the scoring (7) between the flanges. The step of rupturing the scoring further and pulling the sheath (6) out of the port has not begun in FIG. 5.

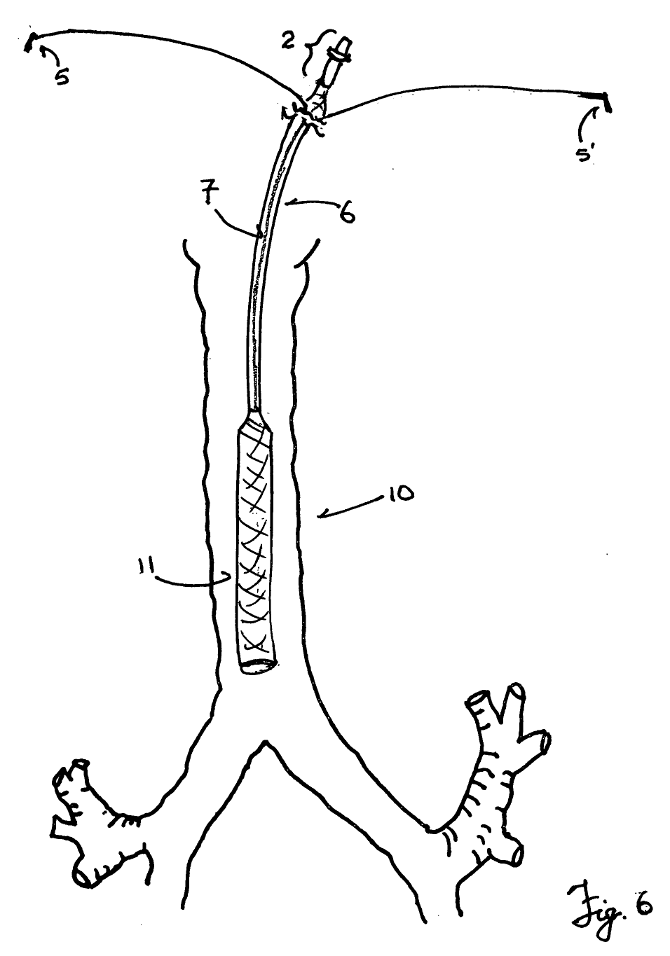

FIG. 6 shows a cuffless, hubbed, tear-away sheath, NaPier ET tube inserted in the trachea (10), after about half of the sheath (6) has been ruptured and withdrawn through the port by pulling on the sheath flanges (5,5′) with hub (2) held stationary, with activation of a corresponding length of the shape-memory element (11). The sheath (6) has lubricity, e.g., by being constructed of or coated with PTFE, and the compressed (unactivated portion) shape-memory element has longitudinal rigidity so that pulling the sheath out through the port does not cause the shape-memory element to move proximally so long as the hub (2) is stationary. The rupture propagates distally down the two scorings as the operator pulls the sheath flanges laterally away from the hub. The operator pulls the two ruptured halves of the sheath out of the passage, which allows the shape-memory element to expand to its memorized diameter.

A second method of rupturing the removable sheath uses a sleeve with two “sleeve flanges” that are aligned with two sheath flanges on the proximal end of the removable sheath. Each sheath flange is affixed to the proximal portion of a half-circumference of the removable sheath by a base that overlaps the distal portion of the sleeve. The sheath has full-length, longitudinal scoring at the two points where the two sheath flanges abut each other. When an operator simultaneously compresses each sleeve flange and the sheath flange with which a given sleeve flange is aligned, the base of the each sheath flange acts as a lever to rupture the sleeve along the scorings. The rupture propagates distally down the two scorings as the operator pulls the sheath flanges laterally away from the hub. The operator pulls the two ruptured halves of the sheath out of the passage, which allows the shape-memory element to expand to its memorized diameter. Other methods of constructing the hub and of rupturing the sheath are possible. One additional method, the pull-string sheath, is described below.

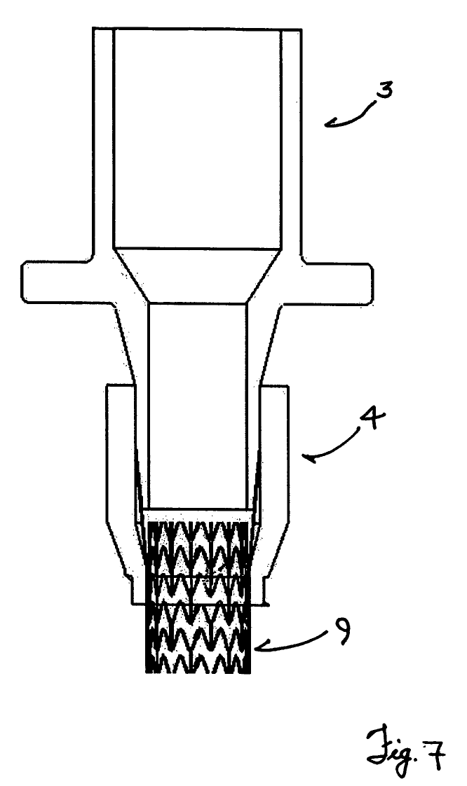

FIG. 7 shows a longitudinal cross-section of the proximal portion of a hubbed, activated NaPier tube. The tear-away sheath has been ruptured and completely removed by the operator through the port of the passage. Expansion of the portion of the shape-memory element (9) affixed to the sleeve (4) is limited by the binding of the sleeve to the shape-memory element.

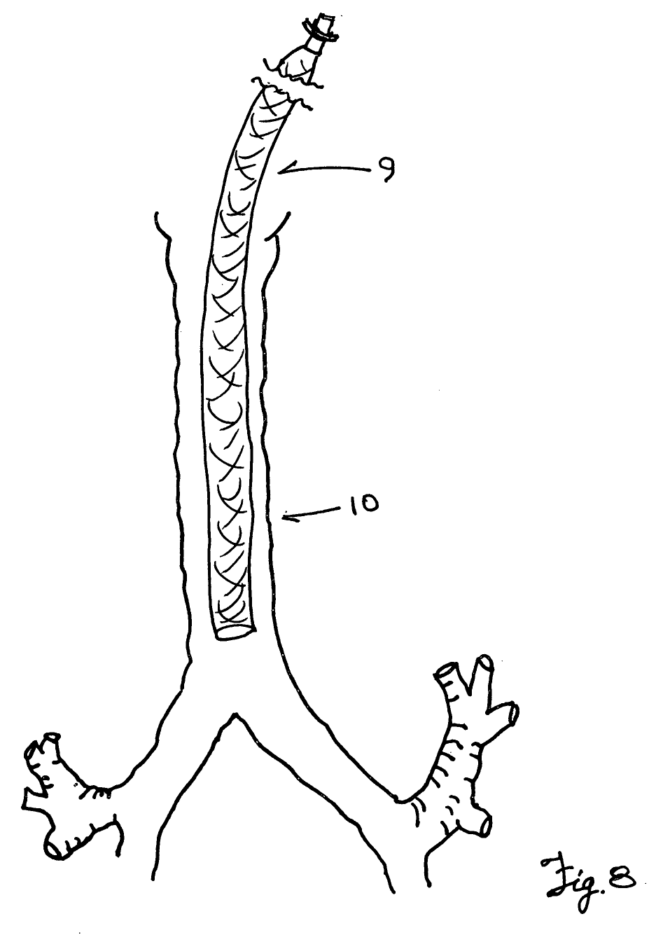

FIG. 8 shows a hubbed, cuffless, NaPier ET tube inserted in the trachea, after activation of the entire length of the shape-memory element. The round, shape-memory element (9) has expanded to its memorized diameter, thereby also stretching the wall material to the memorized diameter and creating a larger lumen in the NaPier tube. As a visualization aid in the Figures, the activated shape-memory element is shown with transparent wall material and the lattice patterns of the shape-memory element are approximations of a laser-cut pattern. Many patterns of shape-memory elements, particularly of laser cut nitinol tubes, are known in the art. The activated NaPier ET tube would have activated dimensions according to the memorized geometry of the shape-memory element, except where restrained by the interior lining of a passage.

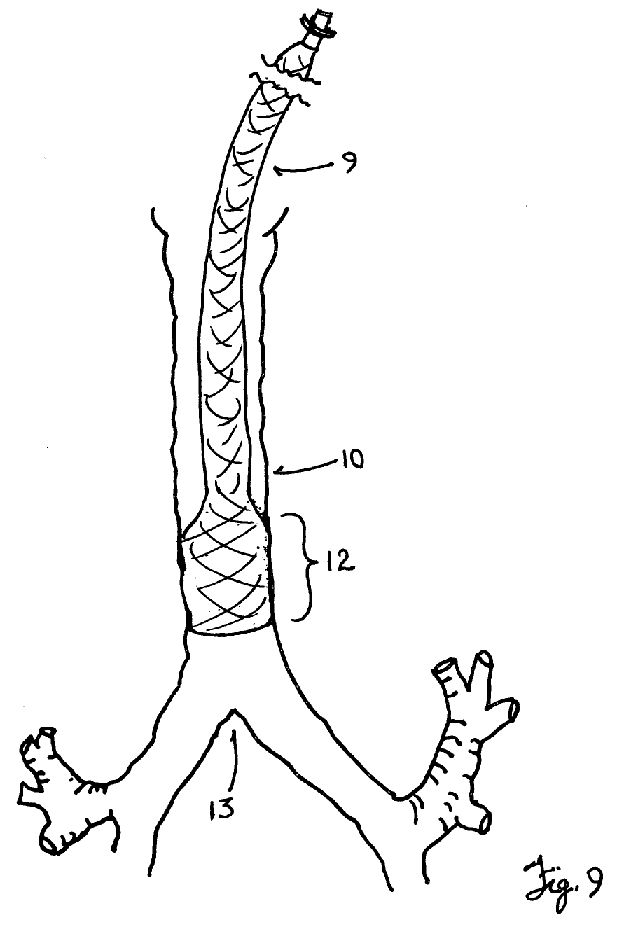

FIG. 9 shows a hubbed, integral cuff, NaPier ET tube inserted in the trachea, after activation of the entire length of the shape-memory element. The integral cuff portion (12) (approximately the last 4 cm of the distal end of the NaPier ET tube) presses tightly against the interior lining of the trachea (10) above the carina (13) to prevent or minimize leakage around the cuff. By selecting a memorized diameter of the shape-memory element, the activated diameter of the activated NaPier tube can expand so that it does not press against the interior lining of the passage, does press lightly against the interior lining of the passage, or presses firmly against the interior lining of the passage. The portion of the integral cuff NaPier ET tube that is proximal to the cuff has a smaller activated diameter. The smaller activated diameter is large enough for mechanical ventilation of a patient under general anesthesia, but smaller enough not to damage the vocal cords of the patient.

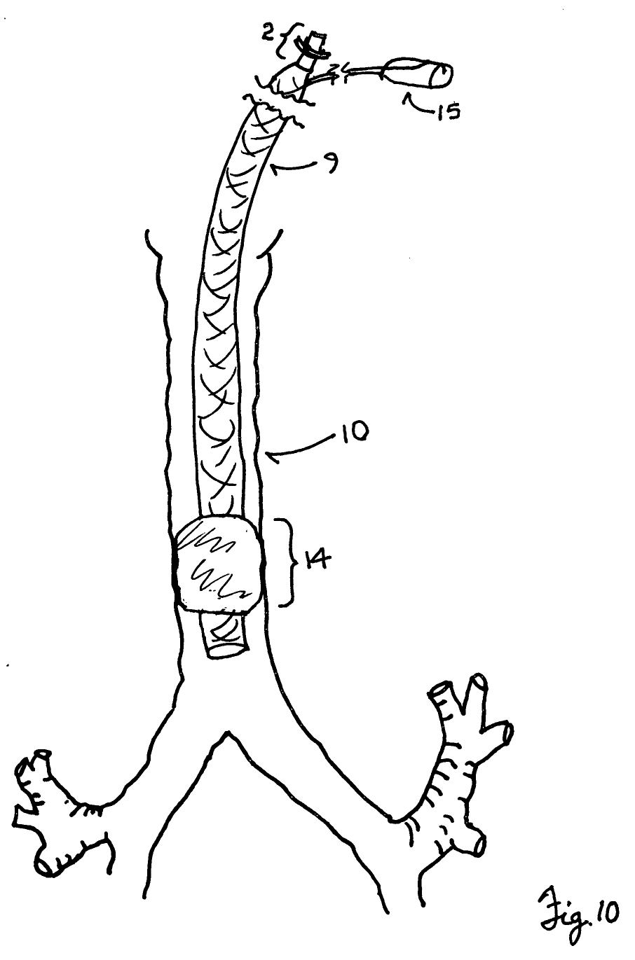

FIG. 10 shows a hubbed, inflatable cuff, NaPier ET tube inserted in the trachea (10), after activation of the entire length of the shape-memory element and inflation of the inflatable cuff (14). The inflated cuff presses firmly against the interior lining of the trachea to prevent or minimize leakage around the cuff. The inflatable cuff (14) is inflatable via an inflation duct that runs inside the lumen of the larger shape-memory element (9) and emerges just distally from the hub (2) to connect to an inflation duct controller (15). As explained below, the inflation duct can be outside or within the lumen of a NaPier ET tube. In both internal and external inflation duct embodiments, the inflation duct is preferably a small lumen NaPier tube.

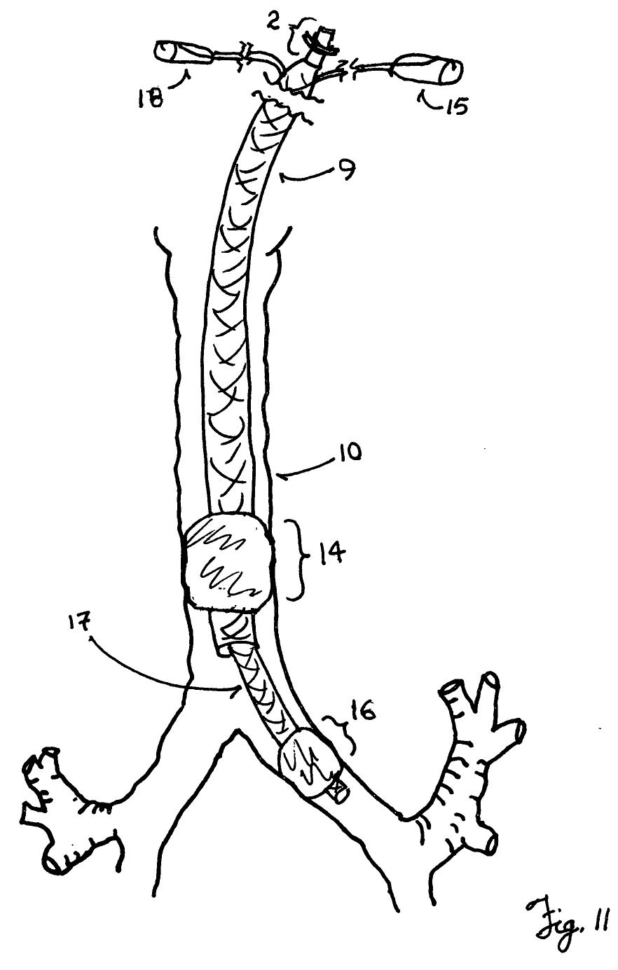

FIG. 11 shows a hubbed, inflatable cuffs, double-lumen, NaPier ET tube inserted in the trachea and one bronchus, after activation of the entire length of the shape-memory element, and inflation of the tracheal inflatable cuff (14) and of a bronchial inflatable cuff (16). The tracheal inflatable cuff (14) is inflatable via an inflation duct that runs inside the lumen of the larger shape-memory element (9) and emerges just distally from the hub (2) to connect to an inflation duct controller (15) for the tracheal inflation cuff. The bronchial inflatable cuff (16) is inflatable via an inflation duct that runs inside the lumen of the second largest shape-memory element (17) and emerges just distally from the hub (2) to connect to an inflation duct controller (18) for the bronchial inflatable cuff. The inflation ducts are preferably small lumen NaPier tubes.

The frame of the NaPier tube is a lattice tube or braid, typically round, of nitinol or of an equivalent non-compliant elastomer or super-elastic alloy. The term “braid” includes a woven braid, a non-woven multi-helix, a coil, a ribbon, and other tubular configurations made of wire or ribbon starting material as opposed a solid tubular starting material. Shape setting (“memorizing”) of a nitinol part is accomplished by deforming (usually in a series of increasing diameters or other dimensions) the nitinol to the shape of a desired component, constraining the nitinol by clamping and then heat treating. Deforming is normally done with the nitinol in a cold-worked condition, for example cold drawn tube or wire. For an activated diameter that is one or more multiples of the starting diameter, shape setting is typically done in iterative, incremental steps of expansion and annealing. Heat treating temperatures for binary nickel-titanium alloys are usually in the range of 325° C. to 525° C. in order to optimize a combination of physical and mechanical properties. Heat treating times are typically 5 minutes to 30 minutes, and the process is well known in the art. Nickel/titanium ratios are substantially 1 part nickel to 1 part titanium in nitinol. The choice between lattice tube and braid depends generally upon how much compressive force an activated tube must withstand and the intended selling price. Lattice tubes of nitinol are typically created by automated laser-cutting and removal of significant portions of the area of the annular wall of the tube, which automated methods are well known in the art. A laser-cut tube is called herein a “lattice tube” or “laser-cut tube”. Broadly speaking, a lattice tube can be manufactured with more resistance to compressive forces than a tube made of braided nitinol wire: the less annular wall area removed, the stronger the resistance to compressive force. Lattice tubes are generally more expensive to produce than braided tubes. Unless otherwise stated, lattice tubes are used in the embodiments of the NaPier tube described below.

At a minimum, a NaPier tube comprises a frame or “shape-memory material”, wall material, and removable sheath. The various combinations of frame, wall material, and sheath are discussed below, followed by a discussion of NaPier tubes for specific uses and how NaPier tubes are used.

The frame of a round, lattice-cut, NaPier tube is preferably laser-cut from a round tube of shape-memory nitinol. The frame's austenitic finish temperature must be below body temperature. When starting with a nitinol tube with smaller diameter than the ultimate activated diameter, after cutting the circumferential pattern, the nitinol frame is expanded and heat treated to be stable at the desired activated diameter (aka “memorizing” the activated diameter or geometry). The heat treatment also controls the transformation temperature of the nitinol such that the frame is typically in its austenitic phase below body temperature.

Alternatively, a thin-wall, nitinol tube with a diameter of the desired activated diameter can be laser-cut with a circumferential pattern; the starting diameter is the memorized or activated diameter. Starting with a thin-wall, round, nitinol tube with a diameter of the desired activated diameter has several advantages, e.g., shorter manufacturing cycle, higher uniformity in the activated geometry, wider choice of thermo-mechanical properties, better fatigue resistance, and better radial strength, and therefore is preferred to starting with a smaller diameter nitinol tube.

The length of the raw nitinol tube is selected to produce a NaPier tube that is, when activated, of the length appropriate for the passage to be intubated. There is typically not a substantial variation in length between a raw nitinol tube and one that has been laser-cut and heat treated, with or without expansion to reach the memorized diameter. The frame is preferably electro-polished to obtain a smooth finish with a thin layer of titanium oxide placed on the surface. An alternative method of making a frame is a tubular braid, typically a ribbon or woven braid of nitinol. The coiled ribbon or tubular braid diameter is equal to the activated diameter; the braid is heat-treated to memorize the activated diameter. The term “prepared frame” means a frame for a NaPier tube prepared as described herein prior to application of wall material. The thickness of the annular wall of a prepared round frame at its activated diameter is typically less than 1.0 mm and preferably ranges from about 0.1 mm to about 0.5 mm. An especially preferred annular wall thickness of a prepared frame is about 0.20 mm to 0.40 mm.

Frames can be made with different rigidity (resilience) and/or different activated geometries in different longitudinal portions of the frame. For instance, the “integral cuff” portion of an integral cuff NaPier ET tube can be made by memorizing a larger activated diameter, e.g., about 25 mm, in about a 4 cm portion of the lower distal portion of the frame (i.e., that portion placed in the trachea), compared with an activated diameter of about 8 mm in the remainder of the frame, as shown in FIG. 9.

To create the annular wall of a NaPier tube in the preferred embodiments, one or more bio-compatible, thermoplastic elastomers are applied to a prepared frame. Various polymers known in the art of bio-compatible elastomers, e.g., polyurethane, parylene, polyethylene (e.g., polytetraflouroethylene, or “PTFE”), and various application methods known in the art of bio-compatible elastomers, e.g., dipping, spray coating, co-extrusion, vacuum diffusion (mandatory for parylene coating), plasma deposition, vapor transfer, taping, and powder coating, can be used to apply the elastomer. High curing temperatures, however, such as that sometimes used for PTFE and polyurethane coatings, can adversely affect mechanical properties of the frame and therefore high temperature curing processes are not preferred. Embodiments of the NaPier tube that use attached fabrics (as opposed to coatings) as wall material, however, do not require high temperature curing and may be used, but typically produce a greater unactivated diameter than elastomeric coatings. Each selected elastomer is applied to the frame, preferably by using a coated mandrel and/or spray-coating, as described below, while the elastomer is molten so that the elastomer uniformly covers the interior and exterior surfaces of the frame, as well as filling the interstices of the frame, but leaves the lumen of the frame unobstructed. The spray-coating, or other deposition of elastomer, can be on only the exterior surface of the frame, on only the interior surface of the frame, or on both the interior and exterior surfaces of the frame, so long as the interstices of the frame are filled with elastomer.

To facilitate insertion of instruments in the lumen, to reduce pneumatic drag in the lumen, and to reduce trauma to the passage upon insertion and removal of a NaPier tube, it is preferred that both exterior and interior surfaces of the frame be coated with an elastomer, preferably a lubricous elastomer. One economical method of applying elastomeric wall material is spray-coating the interior and exterior surfaces of a prepared frame, followed by curing. To obtain the smoothest interior surface of a NaPier tube, one method is (i) to apply elastomer to the contact surface of a cylindrical mandrel or similar form before the prepared frame is placed on the mandrel or form; (ii) to place a prepared frame on the mandrel or similar form, thereby coating the interior surface of the annular wall of the frame; and (iii) to spray-coat the exterior surface of the prepared frame while the frame is mounted on the mandrel. The elastomer is typically cured while on the mandrel or form, and the coated frame removed after curing. Dipping a prepared frame in molten elastomer to apply elastomer is less preferred, since dipping can produce an irregular surface coating and thickness. If vacuum deposition or plasma deposition is used to apply elastomer to the frame, multiple iterations of deposition are typically be required to build up an acceptable strength (thickness) of elastomeric coating. The term “coated frame” means a prepared frame that has been coated with elastomer(s) and the elastomers cured, or a fabric barrier attached to the exterior of the longitudinal surface of the frame, thereby creating the wall of a NaPier tube, as described in this and the following paragraphs.

The elastomers applied to the frame are selected to provide an abrasion and puncture resistant, airtight, annular wall with adequate hoop strength and softness. Since the frame sets the activated diameter, compliant elastomers, such as polyethylene or polyolefin copolymers, silicone polymers, and polyether glycol/polybutylene terephthalate block copolymers, are preferred. The exact “activated diameter” is the memorized diameter of the frame plus twice the thickness of the exterior coating (or the diameter of the activated or inflated cuff, in the cuff portion of a cuffed NaPier tube). Generally speaking, compliant (continuously distensible) elastomers have high pin-hole resistance, but tend to form thick layers. Non-compliant (distensible to a limit) elastomers tend to form thin layers and have poor pin-hole resistance. Compliant elastomers provide a degree of softness to the tube, which aids its navigation of passages with minimal trauma. If additional hoop strength is required, for instance when frames are made with nitinol wire or braid, a non-compliant elastomer (e.g., polyethylene terephthalate (PET) or a polyamide) can be used instead of, or preferably in addition to, a compliant elastomer. A compliant elastomer may be combined with a non-compliant polymeric material as an outer elastomeric layer disposed upon an inner layer of the non-compliant polymeric material, as both an inner elastomeric layer and an outer elastomeric layer disposed upon an intermediate layer of the non-compliant polymeric material, or as a blend of the compliant elastomer and the non-compliant polymeric material. The descriptions of U.S. Pat. No. 6,136,258 (Wang) and U.S. Pat. No. 6,086,556 (Hamilton) are hereby incorporated in full by reference.

Cuffless NaPier ET tubes are particular suited for emergency intubations, such as at accident scenes or in combat zones, where endoscopes are not available. Since an unactivated, cuffless NaPier ET tube can be less than half the diameter of a traditional endotracheal tube, intubation is much safer and easier than with traditional endotracheal tubes. Moreover, NaPier tubes can be compressed during manufacture so that there is no lumen in the unactivated tube, e.g., for use without endoscopic navigation. Such smaller diameter NaPier ET tubes provide even greater safety and ease of use, especially for untrained operators.

PTFE coatings impart a desirable lubricity: an exterior PTFE coating facilitates sliding a NaPier tube into a passage; an interior PTFE coating facilitates movement of an endoscope and surgical instruments in the lumen of the tube and reduced aerodynamic friction during ventilation. A preferred embodiment of the NaPier tube uses PTFE as the sole elastomeric coating on a nitinol frame. A solely PTFE-coated frame is advantageous for plain (cuffless) NaPier ET tubes, integral cuff NaPier ET tubes, and inflatable cuff NaPier ET tubes, but presents a challenge in bonding an inflatable cuff to a PTFE-coated tube. One method of constructing a solely PTFE-coated frame is to coat the exterior of the frame by spray coating, vacuum diffusion, plasma deposition, or other methods known in the art, evaginate the frame, and coat the former interior surface by spray coating, vacuum diffusion, plasma deposition, or other methods known in the art; the former interior surface, after evagination, is the exterior surface.

Attaching an inflatable cuff (e.g., a polyurethane cuff) to a PTFE coated NaPier ET tube is typically done by using a cuff with microperforations in the proximal and distal margins of the cuff (i.e., where the cuff is to be affixed to the exterior wall of the tube) and an inflation duct that communicates longitudinally from the cuff to the port. The inflation duct is either a smaller diameter NaPier tube or a polymer tube known in the art of inflation ducts. The inflation duct is connected to the inflatable cuff using methods known in the art. If a polymer tube is used to construct the inflation duct to be used with a PTFE coated NaPier tube, the inflation duct is typically constructed with two longitudinal flanges with microperforations in the flanges. PTFE is deposited into the microperforations on the cuff margins (and on the duct flanges of a polymer inflation duct); the coating of the cuff margins (and inflation duct flanges) bonds the inflatable cuff (and inflation duct) to the PTFE-coated tube. Inflation pressure applied to the inflation duct inflates any compressed lumen areas of the inflation duct and also inflates the inflation cuff. If a smaller NaPier tube is used as the inflation duct, the NaPier tube is constructed as described below for a double-lumen NaPier tube. An inflation duct can be constructed either in the lumen of a NaPier tube or on the exterior surface of a NaPier tube. The preferred method is to construct a minor duct (e.g., an inflation duct, dispensing duct, suction duct (described below)) in the lumen of the NaPier tube so that a uniform exterior surface is presented to the respiratory tract during intubation and later removal of a NaPier ET tube. Other methods of attaching an inflatable element e.g., an inflatable cuff (and inflation duct), to a PTFE coated substrate, e.g., a shape-memory tube with PTFE annular wall material, are known in the art, e.g., treatment of a PTFE surface with sodium naphthalene to remove fluoride atoms from the PTFE polymer chains, followed by application of adhesives such as rubberized methyl methacrylates; with or without scuffing or plasma etching the PTFE surface to provide a better purchase for adhesives.

The cured coating of one or more elastomers essentially encase the wall of the frame in a uniform, airtight, elastic coating, but leaves the proximal and distal openings of the frame open and the lumen of the frame unobstructed. Therapeutic drugs, agents or compounds may be mixed with or applied to the elastomers in at least a portion of a wall of a NaPier tube. Therapeutic drugs, agents or compounds may be selected to reduce a subject's reaction to the introduction of a NaPier tube to the subject (especially for long term intubation, e.g., of comatose patients). Lubricious polymers (such as PTFE and crystalline polymers) are less porous, more lubricious, and more easily handled through the compression and insertion processes used to insert a coated frame into a sheath, and lubricious polymers are therefore preferred. Coating interior and exterior surfaces of a prepared frame produces a coated frame with an annular wall thickness of preferably 1 mm or less. Thicker coatings (e.g., about 1 mm total), however, are less likely to have pinholes and are more resistant to pinhole propagation.

In hubbed NaPier tubes, the proximal end of the frame is preferably not coated with elastomer(s) before the hub is attached to the frame. Preferably, after all but the proximal end of the frame has been coated with elastomer(s) and the elastomeric coating has cured, to attach the hub to the coated frame, the annular wall of the proximal end of the frame, in its activated diameter, is coated with adhesive and fitted in an annular slot in the sleeve of the hub, and the adhesive allowed to set. Alternatively, the entire frame can be coated with elastomer(s) as described above, the elastomeric coating cured, and the proximal end of the frame bonded to the hub, typically by coating the annular wall of the proximal end of the frame, in its activated diameter, with adhesive and fitting the proximal end in an annular slot in the sleeve of the hub, and the adhesive allowed to set. The second method is better adapted to production systems that produce long, uncut lengths of coated frame, and laser-cutting lengths of coated frame as needed to match demand for such lengths. Regardless of whether the first or second method is used, the distal edge of the wall of the distal end of a coated frame is also preferably coated with elastomer, using one of the methods described above, and the adhered elastomer cured; coating the distal end reduces the risk of tissue damage inflicted by the somewhat blunt edges of the distal end of the frame. In producing fixed lengths of frame, e.g., for NaPier ET tubes, the distal surface of the wall of the distal end of a coated frame is preferably coated concurrently with the remainder of the frame. Hub parameters (strength, diameter, etc.) and fittings (ventilator, etc.) adapted to a given passage being intubated are no different for NaPier ET tubes than for NaPier port-access tubes, unless noted below.

An alternative method of constructing a Napier tube is to first construct an elastomeric (e.g., polyurethane) tube with a diameter barely large enough to accept a compressed, prepared frame, to insert the prepared frame into the elastomeric tube, and to insert the tube and frame into a sheath. Another alternative method of constructing a Napier tube is to first construct an elastomeric (e.g., polyurethane) tube with a diameter barely large enough to accept a compressed, prepared frame, to insert the tube into a sheath, and then to insert the prepared frame into the sheathed, elastomeric tube. The preceding two methods produce a NaPier tube that lacks elastomer on the interior wall of the prepared frame. Such embodiments are less expensive to manufacture, but are not recommended for long term use, since the interstices between the uncoated frame and elastomeric tube can become pathogen reservoirs.

A coated and cured frame is inserted into a sheath, typically by guiding the coated frame through a wide entry aperture to a narrow exit aperture of a conical channel; the proximal, open lumen, end of the sheath is abutted to the exit aperture and dimension such that the entry aperture of the sheath is no narrower that the exit aperture of the conical channel. Compression of the nitinol mesh can also be facilitated by cooling the coated frame, even cryogenically, and inserting the coated tube in a sheath. For instance, Machine Solutions, Inc, (www.machinesolutions.com) sells a machine that compresses various types of shape-memory material.

The sheath is made of flexible, resilient elastomer, such as polyvinyl chloride-based polymers, polyurethane-based polymers, polyethylene-based polymers, polytetraflouroethylene, or other polymers known in the art of catheters and of sheaths for stents. Such elastomers may include co-polymers, fillers in the polymer resin, or side groups on the polymer monomer to add hardness and to enhance the ability of the sheath to propagate a crack along a scored surface, or to be sheared by a pull-string, as described below. The proximal and distal ends of the sheath typically have open lumens for use of a NaPier tube with an endoscope; alternatively, the distal end of the sheath can be in the form of a blunt point if an endoscope is not to be used with the NaPier tube. In the preferred embodiment, the annular wall of the sheath is longitudinally scored from its proximal end to its distal end; the scoring is typically in two places 180° apart on the sheath circumference and is laterally deep enough to facilitate rupturing the sheath into two longitudinal halves. The longitudinal scoring of the sheath is typically done as part of the extrusion of the sheath elastomer during manufacturing of the sheath. At the proximal end of the sheath, a lever is integrally associated with each tear-away half.

Upon placement of the distal end of a NaPier tube in the target location, compression by the operator of the proximal levers, twisting by the operator of the hub to thread the hub into the sheath flange threads, or employing other mechanisms known in the art, causes a crack to propagate down the scoring in a proximal to distal direction as the halves of the sheath are separated and withdrawn; the withdrawal of the two halves of the sheath allows the compressed NaPier tube to expand to its activated diameter in a distal to proximal direction. This activation means is known as a “tear-away sheath”. Sheaths made of highly resilient polymer may require both internal and exterior scoring to facilitate severing the sections of the sheath for removal. The coated frame, now unrestrained by the sheath, expands to its memorized diameter or until resistance from contact with the passage lining equals the radial force of the activated frame. The separated halves of the sheath are easily withdrawn from the passage by pulling the sheath halves from the proximal end, initially by the levers and later, as necessary, by successively gripping and pulling the halves of the sheath withdrawn from the passage. Depending on the elastomer used and the length of the sheath, the distal end of the sheath terminating in a blunt distal point can be manufactured so that the distal point is deeply internally scored at the point to enhance rupturing of the halves at the distal end. A probe inserted in the tube lumen can also be used to rupture the scoring on the distal end of a closed end sheath. More than two longitudinal scorings, each associated with a proximal lever, can be used, especially for sheaths with closed distal ends, to make rupture and withdrawal of the sheath easier.

An activating means other than scoring the sides of the sheath, such as making the sheath with a narrow, soft longitudinal section into which pull-strings are embedded and attaching the pull-strings to pull rings or tabs on the proximal end of the NaPier tube (“pull-string sheath”), can be used to sever the sheath and activate the NaPier tube. In a pull-string sheath, each pull-string is embedded from the proximal end to the distal end of the sheath in a narrow, soft longitudinal section of the sheath; the soft sections, each of which has an embedded pull-string, demarcate the severable sections of the sheath. Each pull-string is folded back 180 degrees at the distal end of the NaPier tube, and is routed at the distal end of the sheath to the exterior of the soft section in which the proximal to distal section of the pull-string is embedded (“corresponding section”). Each pull-string runs from its distal appearance on the exterior of the sheath back to the proximal end of the sheath, and is lightly adhered to the exterior of the corresponding section on that route. To activate NaPier tube equipped with pull-strings, the operator pulls all the pull rings or tabs affixed to the proximal, exterior ends of the pull-strings in a longitudinal direction away from the proximal end of the NaPier tube until all the corresponding sections are severed, and the severable sections of the sheath separate from each other progressively from distal to proximal end. This activation means is known as a “pull-string sheath”.

A pull-string sheath typically has a soft plastic, tabbed “release ring” (like the tabbed ring used to release a cap from a plastic milk jug), the distal end of which tabbed ring is aligned with the proximal end of the embedded pull-strings; the release ring secures the proximal end of each of the embedded pull strings. After the pull-strings are pulled, and the severable sections severed, the tab of the release ring is pulled and the release ring removed, which frees the proximal ends of the severed sections. The operator then pulls the proximal ends of the severed sections, which withdraws the severed portions from the passage. Removing, in a distal to proximal direction, the severed sections of a pull-string sheath activates the NaPier tube (enables the coated frame to expand to its memorized diameter). The tear-away sheath is preferred to the pull-string sheath since the tear-away sheath is less expensive to manufacture and does not cause severed sections of the sheath to be withdrawn through a passage. Other activation means disposed around the circumference of the sheath can be used.

To aid in critical placements, the distal end of a NaPier tube can optionally be made with a radioopaque marker that the operator can observe using a radiographic sensor and display. Means of navigation of a passage other than endoscopic and radiographic, such as the means used for placement of catheters, can be used to place a NaPier tube. When endoscopic navigation is not used, a NaPier tube made with a sheath with a closed distal end is preferred to minimize trauma to the passage lining.

After use by the operator, a NaPier tube is withdrawn from a passage by the operator's pulling on the proximal end of the NaPier tube. Pulling on the proximal end of the tube generates an elongating tensile force that causes the shape-memory element to reduce its diameter, which facilitates withdrawal of the tube. When necessary, a thin, tubular “removal sheath” can be slipped over the full length of the exterior of the tube; as a pulling (tensile) force is applied to the proximal end of the tube (using a tool that grips the proximal end of the tube, if necessary), the lumen width of the shape-memory element decreases at the proximal end. The removal sheath is pushed from the proximal end of the NaPier tube down the exterior of the tube, thereby helping to compress the diameter of the tube as the pulling continues and facilitating removal of the tube, especially an integral cuff NaPier ET tube. The pulling force progressively contracts the more distal portions of the NaPier tube, allowing withdrawal through the port into which the NaPier tube was inserted. A removal sheath is made of flexible, resilient elastomer, such as those described above for tear-away sheaths, but without longitudinal scoring. The distal end of a removal sheath has a conical shape, with an entry (most distal) diameter slightly smaller than the activated diameter of the proximal portion of a NaPier tube. The conical section of the distal end tapers to a lumen diameter to compress the NaPier tube for removal. The smaller (exit) lumen diameter of the conical section is typically a few millimeters smaller than the average lumen diameter of the activated NaPier tube. To use a removal sheath to remove a hubbed NaPier tube, the hub of the NaPier tube is cut off to allow the removal sheath to be placed around the proximal end of the NaPier tube.

Rather than using elastomeric coating(s) to construct the annular wall of a NaPier tube, other types of flexible, pliable, and resilient barriers can be used, such as Goretex® or PTFE (e.g., Teflon®) affixed to a prepared frame in ways known in the art, so long as the compression and insertion into a sheath, activation (expansion), and removal of the NaPier tube are not impaired. The elastomeric coating(s) or equivalent barriers are typically impermeable by gas or fluids. Regardless of whether the wall material includes elastomer, woven fabric, or other barrier material, the wall material is selected to be non-adherent to tissue, to decrease the risk of tissue being caught or of puncture of the passage, and to decrease the risk of tissue growing into the frame. The degree to which gas (e.g., air) or fluid (e.g., blood) permeability can be tolerated, even in miniscule amounts, through a wall of a NaPier tube depends upon the procedure and/or passage for which the NaPier tube is used. For instance, the annular wall of NaPier ET tubes, and NaPier tubes used in port-access surgery, must be both gas and fluid impermeable.

A NaPier ET tube can also be made with a longitudinal duct (“dispensing duct”) between the proximal end and a point near the distal end for administration of nebulized anesthetic and/or medicament. The art of embedding or affixing a dispensing duct is substantially the same as that for inflation ducts, other than the distal terminus of the duct is open to the lower trachea.

A variation on a NaPier ET tube with a dispensing duct is a double-lumen NaPier tube. A double-lumen NaPier tube is used for intra-thoracic surgery, e.g., lung surgery, and for endoscopic surgery elsewhere in the body. A double-lumen embodiment for lung surgery allows one-lung to be ventilated while the other lung can be collapsed to make surgery easier. The second lumen is created by a second, smaller, coated frame disposed within a larger coated frame. In such an embodiment for lung surgery, bronchial cuffs are used instead of, or in additional to, a tracheal cuff. Bronchial cuffs are constructed using the same principles used for inflatable cuff, and integral cuff, NaPier ET tubes, respectively, as described above. In such an embodiment for lung surgery, the second coated frame emerges from the coated frame in the distal portion, and the bifurcation formed by the first and second coated frames is placed slightly above the carina. After being navigated in parallel into the trachea, the first and second coated frames can be independently navigated using separate endoscopes into the right and left main bronchi. The second, smaller coated frame is typically bonded longitudinally to a small portion of the arc of the interior wall of the larger coated frame. The bonding of the smaller coated frame is typically by the same coating and coating process used to coat the larger and smaller prepared frames, as follows: all but the longitudinal point of contact of the frames is coated, the smaller tube inserted inside the larger tube and aligned, then the point of contact is coated, thereby sealing the annular walls of both the larger and the smaller tubes and bonding the smaller tube to the larger tube. Other methods, e.g., adhesive-based, of affixing the smaller coated frame inside a larger coated frame can be used. In cases where a manufactured double-lumen NaPier ET tube is not available, a smaller, cuffless, Napier tube can simply be inserted inside a larger, cuffed Napier tube.

Other double-lumen Napier tubes can be constructed in which the distal portions of the first and second coated frames are not bifurcated. In the same manner as a double-lumen NaPier tube is constructed, NaPier tubes with three or more frames can be constructed and navigated using endoscopes. Multiple frame NaPier tubes, also called “multiple-lumen NaPier tubes”, since each frame provides a lumen, are particularly useful in endoscopic surgery, e.g., one lumen can be used for suction, another lumen for lighting, and another lumen for surgical instruments. The complexity of fittings at the hub typically limits the number of lumens inside the largest diameter NaPier tube in a multiple-lumen NaPier tube to ten lumens. NaPier tubes of smaller diameters than the largest diameter NaPier tube are affixed inside the largest diameter NaPier tube and comprise the additional lumens.

The emergence of smaller diameter NaPier tube through the wall of a larger diameter NaPier tube, such as at the bifurcation in the distal portion of a double-lumen NaPier tube for endoscopic lung surgery, typically requires welding (e.g., laser, plasma, resistance, or e-beam welding) of the frames at the point of emergence. The laser cut pattern of the frames includes a special pattern in the area of the welding. The frames to be used in welding are typically coated with wall material except in the area of welding, the frames are welded, and the area of welding is then coated with wall material.

In hubbed, double-lumen NaPier tubes, the second tube typically has a separate appearance in a fitting in the proximal end of the hub where a hose connects the second tube fitting with a ventilator, pump, or other device; alternatively, the shape-memory material and wall material comprising the smaller tube can emerge, near the proximal end of the NaPier tube, from the interior of the first tube as a separate tube, and terminate at the proximal end of the separate, second, tube in a fitting for connection to a ventilator, pump, or other device. Similarly, in a NaPier of three or more lumens, the constituent tubes can be constructed to separate in the distal portion and/or in the proximal portion of the tube.

A NaPier ET tube can also be made with a longitudinal duct (“suction duct”) between the proximal end and an opening to the exterior of the NaPier tube (“suction port”) near the proximal end of the cuff for suction and removal of fluid that collects above the cuff. The art of embedding or affixing a suction duct is substantially the same as that for inflation ducts, other than the distal terminus of a suction duct is open, through the suction port, to the interior of the trachea just above the cuff (or to a bronchus, in the case of a bronchial cuff, as described above). Both inflatable cuff and integral cuff embodiments of the NaPier ET tube can be constructed with suction ducts and suction ports. In hubbed NaPier ET tubes, the suction duct typically has a separate appearance in fitting in the proximal end of the hub where a hose connects the suction duct fitting with a suction pump; alternatively, the shape-memory material and wall material comprising the suction duct can emerge, near the proximal end of the larger diameter NaPier ET tube, from the interior of the larger diameter NaPier ET tube as a separate tube, and terminate at the proximal end of the separate “suction tube’ in a fitting for connection to a suction hose.

In a preferred pediatric use, a NaPier ET tube is fabricated with a lumen slightly greater than 1.8 mm in its unactivated state (typically a lumen of 2 mm) and is slid over a 1.8 mm pediatric laryngoscope or bronchoscope. For uses with adult patients, larger diameter NaPier ET tubes, and larger laryngoscopes or bronchoscopes, can be used. The patient is typically awake (not under general anesthesia) and typically in a sitting position when intubated. The NaPier ET tube, with an endoscope in the lumen of the tube, is placed into the trachea via a transnasal or transoral route after local anesthesia, e.g., Xylocaine nebulization and bilateral topical 4% Xylocaine and ¼% Neosynephrine to the nasal cavity.

Generally speaking, the diameter of the axial lumen of an unactivated NaPier tube is selected so that the lumen is slightly larger (typically, less than 0.5 mm) than the outside diameter of the endoscope to be used for navigation of a given passage, and the activated diameter is selected based on the passage diameter and procedure to be conducted. In alternative embodiments, a NaPier tube with several different activated diameters can be manufactured by memorizing different diameters or shapes (geometries) along the length of the tube, typically for use in endoscopic surgery, such as laparoscopic surgery. In these port-access NaPier tube embodiments, one or more Napier tubes provide a surgical cannula through which a lens and lighting system and various surgical instruments reach the operative field. The lengths and geometries of cannulae for endoscopic surgery are well known in the art. By memorizing a specific, non-circular “holding section” shape in the mid-portion of a coated frame, a NaPier tube that, when activated, has a reservoir or holding section can be manufactured for use in, e.g., a cholecystectomy with an insufflated abdominal cavity; gallstones collected from the gall bladder can be temporarily deposited in the holding section of the NaPier tube, and the holding section periodically cleaned with a cleaning instrument.

In another embodiment, the NaPier ET tube is fabricated so that the unactivated NaPier ET tube fits through the biopsy port of an endoscope. This embodiment also has two configurations, cuffless and cuffed, as described above. In use, one of these embodiments is inserted though the biopsy port of a bronchoscope or esophagoscope and pushed down the biopsy channel until the distal end of tube reaches the desired depth, typically at the bottom of the trachea. After placement in the trachea, the NaPier tube is activated as the bronchoscope is progressively withdrawn from the tracheal, laryngeal, hypopbaryngeal, pharyngeal, and nasotracheal (or oratracheal) regions. If an cuffed configuration is used, the cuff is inflated or activated to provide an air seal in the tracheal region.

Those skilled in the art will understand how other embodiments and uses of the invention for other natural and surgically created ports and passages can be practiced without undue experimentation. The length and memorized geometry of a NaPier port-access tube depends upon the length and width of the passage to the intubated; such dimensions are well known in the art. Single-lumen and multiple-lumen NaPier tubes can be constructed for many different endoscopic procedures, e.g., examination, diagnosis, anesthesia, surgery, and therapy, of the alimentary canal, ureter, urethra, prostate, uterus, ear canal, Eustachian tube, intra-thoracic region, and intra-peritoneal region. While the invention has been described specifically with reference to a small number of embodiments, various changes and modifications may be made within the full and intended scope of the appended claims.

I claim:

1. A shape-memory port-access tube constructed of shape-memory material, wall material, and a removable sheath, wherein the wall material is applied to the shape-memory material and the shape-memory material is compressed and inserted into the removable sheath.

2. A shape-memory port-access tube constructed of shape-memory material, wall material, and a removable sheath, wherein wall material is applied to the shape-memory material and the shape-memory material is compressed and inserted into the removable sheath, and wherein the tube's length and memorized geometry are those of an endotracheal tube.

3. The tube of claim 1 or 2, wherein the distal portion of the tube has an integral cuff.

4. The tube of claim 1 or 2, wherein the distal portion of the tube has an inflatable cuff that communicates with the proximal end of the tube via an inflation duct.

5. The tube of claim 1 or 2, wherein the proximal portion of the tube terminates in a hub.

6. A shape-memory port-access tube constructed of shape-memory material, wall material, and a removable sheath, wherein wall material is applied to the shape-memory material and the shape-memory material is compressed and inserted into the removable sheath, and wherein the tube's length and memorized geometry are those of a port-access tube for endoscopic surgery.

7. A circumferentially self-expanding port-access tube constructed of round, tubular, shape-memory material, annular wall material, and a removable sheath, wherein wall material is applied to the shape-memory material and the shape-memory material is compressed and inserted into the removable sheath, and wherein the tube's length and memorized diameter are those of an endotracheal tube.

8. The port-access tube of claim 7, wherein the distal portion of the tube has an integral cuff.

9. The port-access tube of claim 7, wherein the distal portion of the tube has an inflatable cuff that communicates with the proximal end of the tube via an inflation duct.

10. The port-access tube of claim 8, wherein the portion of the tube proximal to the integral cuff has a suction duct that communicates from a suction port near the proximal end of the cuff to the proximal end of the tube.

11. The port-access tube of claim 9, wherein the portion of the tube proximal to the inflatable cuff has a suction duct that communicates from a suction port near the proximal end of the cuff to the proximal end of the tube.

12. The tube of claim 7, 8, 9, 10, or 11, wherein the proximal portion of the tube terminates in a hub.

13. A circumferentially self-expanding, double-lumen, port-access tube constructed of two round, tubular frames of shape-memory material, wall material, and a single removable sheath, wherein wall material is applied to each frame of shape-memory material and the shape-memory material is compressed and inserted into the removable sheath, and wherein the tube's length and memorized diameter are those of a port-access tube for endoscopic surgery, and the two frames can optionally bifurcate in the distal portion of the tube.

14. The port-access tube of claim 13, wherein the distal portion of at least one frame has an integral cuff.

15. The port-access tube of claim 13, wherein the distal portion of at least one frame has an inflatable cuff that communicates with the proximal end of the tube via an inflation duct.

16. The port-access tube of claim 14, wherein the portion of the frame proximal to the at least one integral cuff has a suction duct that communicates from a suction port near the proximal end of the cuff to the proximal end of the tube.

17. The port-access tube of claim 15, wherein the portion of the tube proximal to the at least one inflatable cuff has a suction duct that communicates from a suction port near the proximal end of the cuff to the proximal end of the tube.

18. The tube of claim 13, 14, 15, 16, or 17, wherein the proximal portion of the tube terminates in a hub.

19. A self-expanding port-access tube constructed of tubular, shape-memory material, wall material, and a removable sheath, wherein wall material is applied to the shape-memory material and the shape-memory material is compressed and inserted into the removable sheath, and wherein the tube's length and memorized geometry are those of a cannula for endoscopic surgery.

20. A self-expanding, multiple-frame, port-access tube of up to ten constituent frames and a single removable sheath, wherein each frame is constructed of a tubular frame of shape-memory material and wall material, and wherein wall material is applied to each frame of shape-memory material and the frames are compressed and inserted into the removable sheath.

21. A circumferentially self-expanding, multiple-lumen, port-access tube of up to ten constituent frames and a single removable sheath, wherein each frame is constructed of a round, tubular frame of shape-memory material and wall material, and wherein wall material is applied to each frame of shape-memory material and the frames are compressed and inserted into the removable sheath.

22. A self-expanding endotracheal tube constructed of round, tubular, shape-memory material, annular wall material, and a removable sheath, wherein wall material is applied to the shape-memory material and the shape-memory material is compressed and inserted into the removable sheath.

23. The endotracheal tube of claim 22, wherein the distal portion of the tube has an integral cuff.

24. The endotracheal tube of claim 22, wherein the distal portion of the tube has an inflatable cuff that communicates with the proximal end of the tube via an inflation duct.

25. The endotracheal tube of claim 23, wherein the portion of the tube proximal to the integral cuff has a suction duct that communicates from a suction port near the proximal end of the cuff to the proximal end of the tube.

26. The endotracheal tube of claim 24, wherein the portion of the tube proximal to the inflatable cuff has a suction duct that communicates from a suction port near the proximal end of the cuff to the proximal end of the tube.

27. The tube of claim 19, 20, 21, 22, 23, 24, 25, or 26, wherein the proximal portion of the tube terminates in a hub.

28. The tube of claim 1, 2, 6, 7, 13, 19, 20, 21, or 22, wherein the shape-memory material is selected from the group consisting of nickel/titanium alloy, shape-memory polymer, stainless steel, Cu/Zn/Al alloy, and Cu/Al/Ni alloy.

29. The tube of claim 1, 2, 6, 7, 13, 19, 20, 21, or 22, wherein the wall material is selected from the group consisting of polyurethane, parylene, polyethylene, and polytetraflouroethylene.

30. The tube of claim 1, 2, 6, 7, 13, 19, 20, 21, or 22, wherein the wall material comprises a combination of a compliant and a non-compliant elastomer.

31. The tube of claim 1, 2, 6, 7, 13, 19, 20, 21, or 22, wherein the wall material is selected from the group consisting of polyurethane and polyethylene, and wherein polytetraflouroethylene is applied to the exterior of the wall material, and optionally to the interior of the tube lumen, using a process selected from the group consisting of plasma deposition, spray coating, and vacuum diffusion.

32. The tube of claim 1, 2, 6, 7, 13, 19, 20, 21, or 22, wherein the removable sheath is constructed of polymers selected from the group consisting of polyvinyl chloride-based polymers, polyurethane, polyurethane-based polymers, polyethylene, polyethylene-based polymers, polytetraflouroethylene, and polytetraflouroethylene-based polymers.

33. The tube of claim 1, 2, 6, 7, 13, 19, 20, 21, or 22, wherein the tube is constructed with length and geometry adapted for endoscopic procedures selected from the group comprising alimentary canal, respiratory tract, ureter, urethra, prostate, uterus, ear canal, Eustachian tube, intra-thoracic region, and intra-peritoneal region.
